SEQUENCE LISTING (37 sequences)

SEQ ID NO: 1 - DNA, 20 bp, Artificial Sequence
  Synthetic Sequence
gcttaattaaggtaaacgtc

SEQ ID NO: 2 - DNA, 20 bp, Artificial Sequence
  Synthetic Sequence
ccaacctggcggctcgttgg

SEQ ID NO: 3 - DNA, 20 bp, Artificial Sequence
  Synthetic Sequence
gagtccgagcagaagaagaa

SEQ ID NO: 4 - DNA, 20 bp, Artificial Sequence
  Synthetic Sequence
gggtggggggagtttgctcc

SEQ ID NO: 5 - DNA, 20 bp, Artificial Sequence
  Synthetic Sequence
gatctctagtgggaagtttc

SEQ ID NO: 6 - DNA, 20 bp, Artificial Sequence
  Synthetic Sequence
gtctgtagaaagaatggaag

SEQ ID NO: 7 - DNA, 21 bp, Mus musculus
taaggtaaacgtcgggttaag

SEQ ID NO: 8 - DNA, 23 bp, Mus musculus
cattggttaaggtaaacgtcagg

SEQ ID NO: 9 - DNA, 23 bp, Mus musculus
gcttaattaaggtaaacgtcggg

SEQ ID NO: 10 - DNA, 23 bp, Mus musculus
tctcacaggaggtaaacgtcagg

SEQ ID NO: 11 - DNA, 23 bp, Mus musculus
actcatatcaggtagacgtcagg

SEQ ID NO: 12 - DNA, 24 bp, Mus musculus
gtatgaagcacggtaaacgtcagg

SEQ ID NO: 13 - DNA, 24 bp, Mus musculus
ctagaattaaggtaaacagtcagg

SEQ ID NO: 14 - DNA, 21 bp, Mus musculus
ctggttacggtaaacgtcagg

SEQ ID NO: 15 - DNA, 23 bp, Mus musculus
ccacaatgaaggtaaacatcagg

SEQ ID NO: 16 - DNA, 22 bp, Mus musculus
acgtcttaaggtaaacgtctgg

SEQ ID NO: 17 - DNA, 24 bp, Mus musculus
gcctctgaacaggtaaacgtctgg

SEQ ID NO: 18 - DNA, 24 bp, Mus musculus
acttgaacgaaggtaaacatcagg

SEQ ID NO: 19 - DNA, 24 bp, Mus musculus
acaaaaattaaggtaaacatctgg

SEQ ID NO: 20 - DNA, 23 bp, Mus musculus
gctgcctgtaggtaaacatcagg

SEQ ID NO: 21 - DNA, 22 bp, Mus musculus
gagaattacagtaaacgtctgg

SEQ ID NO: 22 - DNA, 22 bp, Mus musculus
gatacttgaggtaaacatctgg

SEQ ID NO: 23 - DNA, 24 bp, Mus musculus
ctctaatgtcaggtaaacatcagg

SEQ ID NO: 24 - DNA, 23 bp, Mus musculus
gcgtagtcatggtaaacatcggg

SEQ ID NO: 25 - DNA, 22 bp, Mus musculus
acaaaatagagtaaacgtctgg

SEQ ID NO: 26 - DNA, 24 bp, Mus musculus
gacttacgtaaagtaaacgtctgg

SEQ ID NO: 27 - DNA, 22 bp, Mus musculus
tcccaattaggtaaacatctgg

SEQ ID NO: 28 - DNA, 22 bp, Mus musculus
gatttagaaggcaaacgtcagg

SEQ ID NO: 29 - DNA, 23 bp, Mus musculus
gctagagtagagtaaacgtcagg

SEQ ID NO: 30 - DNA, 23 bp, Mus musculus
gcttttctaggataaacgtcagg

SEQ ID NO: 31 - DNA, 22 bp, Mus musculus
cctttttaaggtaaaagtccgg

SEQ ID NO: 32 - DNA, 23 bp, Mus musculus
ccatatctaaggtaaaagtcagg

SEQ ID NO: 33 - DNA, 24 bp, Mus musculus
gcatactgcatggtaaacatcagg

SEQ ID NO: 34 - DNA, 21 bp, Mus musculus
cttaataaggtagacatcagg

SEQ ID NO: 35 - DNA, 23 bp, Mus musculus
actaaaacatggtaaacatcagg

SEQ ID NO: 36 - DNA, 22 bp, Mus musculus
ggtaatagagataaacgtctgg

SEQ ID NO: 37 - DNA, 24 bp, Mus musculus
gactgtacaaaggtaaaagtcagg

(19) United States
(12) Patent Application Publication
(10) Pub. No.: US 2024/0158778 A1
(43) Pub. Date: May 16, 2024

(54) METHODS FOR ASSESSING SPECIFICITY OF CRISPR-MEDIATED GENOME EDITING

(71) Applicants: THE BOARD OF TRUSTEES OF THE LELAND STANFORD JUNIOR UNIVERSITY, Stanford, CA (US); THE UNIVERSITY OF CHICAGO, Chicago, IL (US)

(72) Inventors: Georgi MARINOV, Redwood City, CA (US); William J. GREENLEAF, Menlo Park, CA (US); Tong WU, Chicago, IL (US); Chuan HE, Chicago, IL (US)

(21) Appl. No.: 18/280,653

(22) PCT Filed: Apr. 1, 2022
(86) PCT No.: PCT/US2022/023136

§ 371 Date: Sep. 6, 2023

Related U.S. Application Data
(60) Provisional application No. 63/172,942, filed on Apr. 9, 2021.

(51) Int. Cl. C12N 15/10; C12Q 1/686

(52) U.S. Cl. CPC C12N 15/102; C12Q 1/686

(57) ABSTRACT

The disclosure pertains to a method for isolating DNA fragments that contain a binding site for a guide-RNA of an RNA-guided endonuclease. The method comprises contacting the RNA-guided endonuclease with a target double-stranded DNA, thereby producing a single stranded DNA (ss-DNA) at the site to which the guide-RNA binds. The ssDNA is then reacted with kethoxal or an analog thereof modified by a chemoselective group, thereby adding the chemoselective group to any unpaired guanine based within the ssDNA. A first binding member of a specific binding pair is linked to the chemoselective group, for example, via a cycloaddition reaction. The DNA is fragmented and the DNA fragments that contain the binding sites for the guide-RNA are enriched using a support that comprises a second binding member of the specific binding pair. The method can be used to identify binding sites for a guide-RNA thereby identifying off-target binding sites.

### CROSS-REFERENCING

This application claims the benefit of U.S. provisional application Ser. No. 63/172,942, filed on Apr. 9, 2021, which application is incorporated by reference herein for all purposes.

### STATEMENT OF GOVERNMENT SUPPORT

This invention was made with Government support under contracts HG009436, HG009442, P50HG007735, RO1 HG008140, and U19A1057266 awarded by the National Institutes of Health. The Government has certain rights in the invention.

### INTRODUCTION

CRISPR-based methods for editing the genome and epigenome have emerged as a highly versatile means of manipulating the genetic makeup and regulatory states of cells. CRISPR technology may have the potential to transform medical practice by enabling direct elimination of pathogenic sequence variants. CRISPR has also become a standard tool for discovery in fundamental biomedical research, for example in its use in high-throughput, massively parallel CRISPR screens.

However, the presence of significant off-target effects for many guide RNAs (sgRNAs), wherein the guide-CRISPR complex likely has biochemical activities at genomic sites that are not perfect matches to the sg RNA, presents a major hurdle to fully realizing this potential. Off-target effects are particularly problematic for medical applications, where risks of negative consequences for a patient's health must be minimized as much as possible.

To this end, numerous approaches have been developed to experimentally map off-target effects genome-wide. Methods such as Digenome-seq look for particular types of cut sites around target sequences in whole-genome sequencing data; however, deep whole-genome sequencing is still quite expensive. Assays such as BLESS, GUIDE-seq, HTGTS, DSBCapture, BLISS, SITE-seq, CIRCLE-seq, TTISS, INDUCE-seq, and CHANGE-seq aim to instead directly map Cas9 cleavage events; however, they all involve some combination of complex and laborious molecular biology protocols and non-standard reagents, and have not been widely adopted as a result. Other methods, such as DISCOVER-seq, which maps DNA repair activity by applying ChIP-seq against the MRE11 protein, as well as earlier applications of ChIP-seq to map catalytically dead dCas9 occupancy sites genome-wide, suffer from background and specificity issues associated with the ChIP procedure. Most recently, long-read sequencing has been adapted to the problem of Cas9 specificity profiling, in the form of SMRT-OTS and Nano-OTS, but the cost of these methods is relatively high while their throughput is comparatively low.

Various computational models have also been trained to predict off-targets genome-wide. However, these exhibit far from perfect accuracy, and thus in many situations, especially within clinical contexts, direct experimental evidence is needed to accurately identify potential unintended effects of CRISPR-based reagents.

Therefore a faster, more accessible, and versatile methods are needed for mapping CRISPR off target sites.

### SUMMARY

Certain embodiments provide a method for isolating within a target DNA a DNA fragment that contains a binding site for a guide-RNA of an RNA-guided endonuclease. The method comprises:
(a) contacting the RNA-guided endonuclease with a target double-stranded DNA, thereby producing single stranded DNA (ss-DNA) at the site to which the guide-RNA binds,
(b) reacting the product of step (a) with or an analog thereof modified by a chemoselective group, thereby adding the chemoselective group to any unpaired guanine base within the ssDNA,
(c) linking a first binding member of a specific binding pair to the chemoselective group added in step (b),
(d) fragmenting the DNA after step (c) to produce fragmented target DNA, and
(e) enriching for fragments that contain the first binding member of the specific binding pair by binding the product of step (d) to a support that comprises a second binding member of the specific binding pair, which specifically binds to the first binding member of the specific binding pair.

These assays can be used in both in vitro and in vivo.

In certain cases, linking the first binding member of the specific binding pair to the chemoselective group can be performed via a cycloaddition reaction.

The target DNA can be a genomic nucleic acid, a mitochondrial nucleic acid; a chloroplast nucleic acid; a plasmid; or a viral nucleic acid.

The RNA-guided endonuclease can be an active nuclease or a dead nuclease.

The DNA fragment that contains the binding site for the guide-RNA can be sequenced, for example, via a next generation sequencing method.

Contacting the RNA-guided endonuclease with a target DNA can be performed when the target DNA is outside a cell or inside a cell.

When the target DNA is inside a cell, ssDNA is typically present in the cell, such as ssDNA produced during active transcription. Therefore, in certain embodiments, these ssDNA regions are identified using a control cell that is contacted with an RNA-guided endonuclease without the guide RNA.

The methods disclosed herein could be used to identify off-target activities of CRIPSR enzymes. These and other advantages and features of the disclosure will become apparent to those persons skilled in the art upon reading the details of the compositions and methods of use, which are more fully described below.

### BRIEF DESCRIPTION OF THE FIGURES

FIG. 1 illustrates how kethoxal modified by a chemoselective group (in this case N₃ kethoxal) can be used to tag sites of Cas9-mediated strand invasion.

FIGS. 2A-2K: CasKAS maps dCas9- and Cas9-mediated strand invasion and cleavage events genome-wide in vitro and in vivo. (A) CasKAS is based on the KAS-seq assay for mapping ssDNA structures. N₃-kethoxal covalently modifies unpaired guanine bases (while having no activity for G bases paired within dsDNA). Strand invasion by Cas9/dCas9 carrying an sgRNA results in the formation of a ssDNA structure, which can be directly identified using N₃-kethoxal. (B) Outline of in vivo and in vitro CasKAS. For in vitro CasKAS, gDNA is incubated with a dCas9/Cas9 RNP, then N₃-kethoxal is added to the reaction; for in vivo CasKAS, cells are transfected with an RNP, then treated with kethoxal. DNA is then purified, click chemistry is carried out, DNA is sheared, labeled fragments are pulled down with streptavidin beads, and sequenced. (C and D) Mapping of dCas9 targets in vitro. (C) Mouse gDNA was incubated with dCas9 RNPs carrying one of two sgRNAs targeting the mouse HOXA locus. Highly specific labeling is observed at the expected target location of each sgRNA. (D) Asymmetric strand distribution of in vitro dCas9 CasKAS reads around the sgRNA target site. (E and F) Mapping of Cas9 targets in vitro. (E) Mouse gDNA was incubated with Cas9 RNPs carrying one of same two sgRNAs targeting the mouse HOXA locus. (F) The distribution of 5′ read ends around targets sites in vitro CasKAS datasets shows direct capture of the intermediate cleavage state (SEQ ID NO: 7). (G) Reproducibility of in vivo dCas9 CasKAS datasets. Shown are RPM values for 500 bp windows centered on the top ˜7,000 predicted target sites for the “sgRNA #1” in two in vitro CasKAS experiments. Off-target sites are color-coded by the number of mismatches relative to the sgRNA. (H) CasKAS requires a moderate sequencing depth of 10-20×10⁶ reads to accurately rank potential off-targets. (I-K) In vitro CasKAS maps Cas9 and dCas9 target sites. (I) Shown are CasKAS experiments with Cas9 and dCas9 and with the EMX1 sgRNA or with no sgRNA (negative control) (J) Assymmetric 5′ end distribution around target sites in dCas9 in vivo CasKAS. (K) In vivo Cas9 CasKAS, a mixture distribution is observed between phased cleavage sites and broader ssDNA labeling.

FIGS. 3A-3H: CasKAS profiles sgRNA specificity genome-wide. (A) Summary of de novo peak calls for sgRNA #1 (using MACS2) (B) CasKAS signal is stronger over predicted off-target sites, but legitimate interactions are also found elsewhere in the genome. (C) CasKAS profile over predicted (by Cas-OFFinder) off-target sites for sgRNA #1 with dCas9 (all such sites and focusing only on the top 100 ranked by dCas9 CasKAS signal). (D) CasKAS profile over peak calls outside predicted (by Cas-OFFinder) off-target sites for sgRNA #1 with dCas9. (E) Determinants of sequence specificity as measured by dCas9 CasKAS (for sgRNA #1)(From top to bottom, SEQ ID NOs:8-37). PAM-distal regions of the sgRNA are less constrained than its PAM-proximal parts. The on-target sgRNA is highlighted in yellow. (F) Active Cas9 signal read profiles can be used to distinguish off-targets associated with cutting from those where only binding occurs. Shown are the same off-target sites as in (E) and the plus-and minus-strand active Cas9 5′ end profiles around the sgRNA. In this case (sgRNA #1), only the on-target site shows a Cas9 CasKAS pattern indicating cleavage; at the other sites even active Cas9 likely only binds but does not cut. A simple cutting score metric (“C-score”) based on multiplying the 5′ end forward- and reverse-strand profiles can be used to quantify cutting vs. binding. (G and H) Comparison between in vitro and in vivo CasKAS signal over predicted off-target sites for the EMX1 sgRNA. In vivo CasKAS is quantified as the difference in read per million (±500 bp of the sgRNA site) between the sgRNA KAS-seq and the no-guide control KAS-seq (“RPM_{diff}). The on-target site is shown in blue.

### DEFINITIONS

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

As used herein, the term “kethoxal or an analog thereof, modified by a chemoselective group” refers to kethoxal or an analog thereof that has been functionalized, particularly at the terminal methyl group of the ethoxy moiety, to include a group that is capable of participating in a reaction to link the kethoxal or the analog thereof to a first member of a specific binding pair. Examples of chemoselective groups are numerous and include: amines and active esters such as an NHS esters, thiols and maleimide or iodoacetamide), as well as groups that can react with one another via 1,3 cycloaddition or Click reaction, e.g., azide and alkyne groups. For example, in some embodiments, the chemoselective group may be thiol reactive. A kethoxal analog includes dicarbonyl analogues described below. In some cases, chemoselective reagents react orthogonally to a first member of a specific binding pair. The term “orthogonally” in this context indicates that the chemoselective agent reacts to a first member in a reaction that does not normally occur in a native cell under natural conditions. In some cases, such orthogonal reaction can occur in living systems without perturbing the system's native biological functions and processes. For example, the kethoxal or analog thereof can carry a functional group that can react orthogonally to biotin or other affinity tags for pull-down at enrichment.

Such groups include azido and alkynyl (e.g., cyclooctyne) groups, although others are known (Kolb et al., 2001; Speers and Cravatt, 2004; Sletten and Bertozzi, 2009). N3-kethoxal is an example of a UDP glucose modified with a chemoselective group, although others are known. A kethoxal analog are capable of covalently reacting with unpaired guanine bases in the same way that Kethoxal does.

In certain cases, a kethoxal analog can have the Formula I as follows:

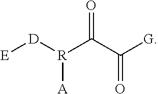

In certain cases, E is selected from a reactive group, click chemistry moiety, binding group, or therapeutic agent; D is a linker; R is a connecting element or group; A is a substituent; and G is a dicarbonyl-defining group. In certain cases, R can be selected from substituted or unsubstituted carbon, nitrogen, aryl, alkylaryl, or heterocyclic group. In certain cases, A can be substituted with one or more (mono-substituted, di-substituted, etc.) of H, F, CF₃, CF₂H, CFH₂, CH₃, alkyl group, or combinations thereof. In certain cases, A can be mono- or di-substituted with a linker. In certain cases, A can be mono- or di-substituted with a reactive group, e.g., a click chemistry moiety, therapeutic agent, or binding moiety.

In certain cases, D is a linker selected from an ester, amide, tetrazine, tetrazole, triazine, triazole, aryl groups, heterocycle, sulfonamide, a substituted or unsubstituted —(CH₂)ₙ— where n is 1-10 with 0, 1, 2, 3, 4, 5, 6, 7, 8, 9, 10 methyl substitutions; —O(CH₂)ₘ— where m is 1-10 with 0, 1, 2, 3, 4, 5, 6, 7, 8, 9, 10 methyl substitutions; —NR⁵— where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶— where k is 1-10 with 0, 1, 2, 3, 4, 5, 6, 7, 8, 9, 10 methyl substitutions and R¹¹ is alkyl, substituted alkyl, cycloalkyl, substituted cycloalkyl, heteroalkyl, substituted heteroalkyl, aryl, substituted aryl, heteroaryl, or substituted heteroaryl. D can be —N(CH₃)—, —OCH₂—, —N(CH₃)COCH₂—, or a group having the chemical formula of Formula VII.

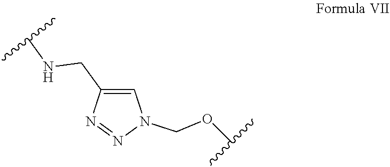 [SMILES: *NCc1cn(CO*)nn1]

In some cases, D can be substituted with a reactive group, e.g., a click chemistry moiety. In some cases, In some cases, D can be a direct bond between E and the carbon atom binding A. In certain cases, D can be a substituent that modulates the stability of the product formed, including alkoxy groups, ethers, carbonyls, aryl groups, electron withdrawing groups (e.g, nitro-, trifluoromethyl-, cyano groups, trimethylsilyl-, esters—either as stand-alone substituents or substituted aryl groups), electron donating groups (e.g, alkyl groups, thiols, amines, aziridines, oxiranes, alkenes—either as stand-alone substituents or substituted aryl groups), electrophilic or nucleophilic centers (e.g, aldehydes, ketones, anhydrides, imines, nitriles, alkenes, alkynes, aryls, heteroaryls), or H-bond acceptors (e.g, ethers, alcohols, carbonyls, amines, thiols, thioethers, sulfonamides, halides).

In certain cases, G can be independently selected from H, CF₃, CF₂H, CFH₂, CH₃, or alkyl group.

In certain cases, E can be selected from alkynes, azides, strained alkynes, dienes, dieneophiles, alkoxyamines, carbonyls, phosphines, hydrazides, thiols, alkenes, diazirines. E can be a substituted alkyl, heteroalkyl, substituted heteroalkyl, heteroaryl, or substituted heteroalkyl. In some cases, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted diazirane, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain cases, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazranes, dienes, dieneophiles, alkoxyamines, carbonyls, phosphines, hydrazides, thiols, and alkenes. In certain cases, E can be further coupled to an agent or binding moiety. In certain cases, the agent or binding moiety binds directly or indirectly to a target (protein or nucleic acid) in vivo, ex vivo or in vitro. In certain cases, the agent or binding moiety binds directly or indirectly to a target (protein or nucleic acid) in vivo.

Specific compounds include, but are not limited to a compound of Formula I where (i) G is H, R is C, A is methyl, D is —OCH₂CH₂-triazole-pyridine-aryl-amide-CH₂CH₂, and E is N₃ (azide); (ii) G is H; R is C, A is F, D is —OCH₂CH₂-triazole-amide-benzoimidazole-phenyl-NHCO—CH₂CH₂, and E is alkyne; (iii) G is H, R is C, A is a di-fluoro substituent of R, D is —OCH₂CH₂-triazole-CH₂-pyridine-benzoimidazole-NHCO—CH₂CH₂CH₂—, and E is N₃ (azide); (iv) G is H, R is C, A is methyl, D is —OCH₂CH₂-triazole-, and E is phenol or diphenol.

In certain cases, a kethoxal analog can have the general formula of Formula II below:

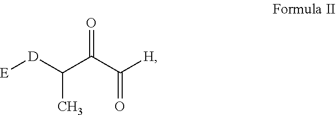

wherein E is selected from a reactive group, click chemistry, binding group, or therapeutic agent; and D is a linker.

In certain cases, D is a linker selected from an ester, amide, tetrazine, tetrazole, triazine, triazole, aryl groups, heterocycle, sulfonamide, a substituted or unsubstituted —(CH₂)ₙ— where n is 1-10 with 0, 1, 2, 3, 4, 5, 6, 7, 8, 9, 10 methyl substitutions; —O(CH₂)ₘ— where m is 1-10 with 0, 1, 2, 3, 4, 5, 6, 7, 8, 9, 10 methyl substitutions; —NR⁵— where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶— where k is 1-10 with 0, 1, 2, 3, 4, 5, 6, 7, 8, 9, 10 methyl substitutions and R¹¹ is alkyl, substituted alkyl, cycloalkyl, substituted cycloalkyl, heteroalkyl, substituted heteroalkyl, aryl, substituted aryl, heteroaryl, or substituted heteroaryl. In some cases, D can be —N(CH₃)—, —OCH₂—, —N(CH₃)COCH₂—, or a group having the chemical formula of Formula VII.

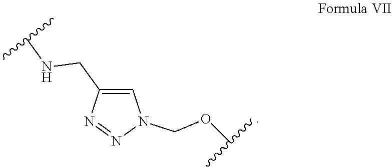 [SMILES: *NCc1cn(CO*)nn1]

In some cases, D can be substituted with a reactive group, e.g., a click chemistry moiety. In some cases, D can be a direct bond between E and the carbon atom binding A. In certain cases, D can be a substituent that modulates the stability of the product formed, selected from alkoxy groups, ethers, carbonyls, aryl groups, electron withdrawing groups (e.g, nitro-, trifluoromethyl-, cyano groups, trimethylsilyl-, esters—either as stand-alone substituents or substituted aryl groups), electron donating groups (e.g, alkyl groups, thiols, amines, aziridines, oxiranes, alkenes—either as stand-alone substituents or substituted aryl groups), electrophilic or nucleophilic centers (e.g, aldehydes, ketones, anhydrides, imines, nitriles, alkenes, alkynes, aryls, heteroaryls), or H-bond acceptors (e.g, ethers, alcohols, carbonyls, amines, thiols, thioethers, sulfonamides, halides).

In certain cases, E is selected from a reactive group, click chemistry, binding group, or therapeutic agent. In certain instances, E can be selected from alkynes, azides, strained alkynes, dienes, dieneophiles, alkoxyamines, carbonyls, phosphines, hydrazides, thiols, alkenes, diazirines. E can be a substituted alkyl, heteroalkyl, substituted heteroalkyl, heteroaryl, or substituted heteroalkyl. In some cases, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain cases, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazranes, dienes, dieneophiles, alkoxyamines, carbonyls, phosphines, hydrazides, thiols, and alkenes. In certain cases, E can be further coupled to an agent or binding moiety. In certain cases, the agent or binding moiety binds directly or indirectly to a target (protein or nucleic acid) in vivo, ex vivo or in vitro. In certain cases, the agent or binding moiety binds directly or indirectly to a target (protein or nucleic acid) in vivo.

In certain cases, a kethoxal analog can have the general formula of Formula III:

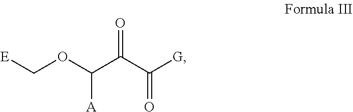

where E is selected from a reactive group, click chemistry moiety, binding group, or therapeutic agent; A is a substituent; and G is a dicarbonyl-defining group. In certain cases, E is a click chemistry moiety selected from alkynes, azides, strained alkynes, dienes, dieneophiles, alkoxyamines, carbonyls, phosphines, hydrazides, thiols, alkenes, and diazirines. In certain cases, E can be selected from alkynes, azides, strained alkynes, dienes, dieneophiles, alkoxyamines, carbonyls, phosphines, hydrazides, thiols, alkenes, diazirines. E can be a substituted alkyl, heteroalkyl, substituted heteroalkyl, heteroaryl, or substituted heteroalkyl. In some cases, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted diazirane, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain cases, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazranes, dienes, dieneophiles, alkoxyamines, carbonyls, phosphines, hydrazides, thiols, and alkenes. E can further comprise a linker (E can be a reactive group having a terminal click chemistry moiety).

In certain cases, A can be a linker, A can be further coupled to an agent or binding moiety. A or G can be independently selected from H, F, CF₃, CF₂H, CFH₂, CH₃, or alkyl group. In certain cases, the agent or binding moiety binds directly or indirectly to a target (protein or nucleic acid) in vivo, ex vivo or in vitro. In certain cases, the agent or binding moiety binds directly or indirectly to a target (protein or nucleic acid) in vivo.

In certain cases, a kethoxal analog can have the general formula of Formula IV:

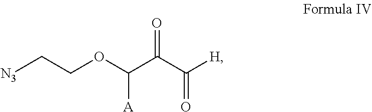

wherein A is substituted with one or more (mono-substituted, di-substituted, etc.) of H, F, CF₃, CF₂H, CFH₂, CH₃, alkyl group, or combinations thereof. In certain cases, A can be mono- or di-substituted with a linker. In certain cases, A can be mono-or di-substituted with a reactive group, e.g., a click chemistry moiety, therapeutic agent, or binding moiety. In certain cases, the azide moiety is further coupled to an agent or binding moiety. In certain cases, the agent or binding moiety binds directly or indirectly to a target (protein or nucleic acid) in vivo, ex vivo or in vitro. In certain cases the agent or binding moiety binds directly or indirectly to a target (protein or nucleic acid) in vivo.

In certain cases, a kethoxal analog can have the general formula of Formula V:

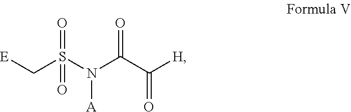

wherein E is selected from a reactive group, click chemistry moiety, binding group, or therapeutic agent, and A is a substituent.

In certain cases, E is a click chemistry moiety selected from alkynes, azides, strained alkynes, dienes, dieneophiles, alkoxyamines, carbonyls, phosphines, hydrazides, thiols, alkenes, and diazirines. In certain cases, E can be selected from alkynes, azides, strained alkynes, dienes, dieneophiles, alkoxyamines, carbonyls, phosphines, hydrazides, thiols, alkenes, diazirines. E can be a substituted alkyl, heteroalkyl, substituted heteroalkyl, heteroaryl, or substituted heteroalkyl. In some cases, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted diazirane, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain cases, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazranes, dienes, dieneophiles, alkoxyamines, carbonyls, phosphines, hydrazides, thiols, and alkenes. In certain cases, E can be further coupled to a linker (E can be a linker having a terminal click chemistry moiety).

A is substituted with one or more (mono-substituted, di-substituted, etc.) of H, F, CF₃, CF₂H, CFH₂, CH₃, alkyl group, or combinations thereof. In certain cases, A can be mono- or di-substituted with a linker. In certain cases, A can be mono- or di-substituted with a reactive group, e.g., a click chemistry moiety, therapeutic agent, or binding moiety. In certain cases, the azide moiety is further coupled to an agent or binding moiety. In certain cases, the agent or binding moiety binds directly or indirectly to a target (protein or nucleic acid) in vivo, ex vivo or in vitro. In certain cases, the agent or binding moiety binds directly or indirectly to a target (protein or nucleic acid) in vivo.

In certain cases, E, A, or E and A can be independently coupled to an agent or binding moiety. In certain cases, the agent or binding moiety binds directly or indirectly to a target (protein or nucleic acid) in vivo, ex vivo or in vitro. In certain cases, the agent or binding moiety binds directly or indirectly to a target (protein or nucleic acid) in vivo.

In certain cases, a kethoxal analog can have the general formula of Formula VI:

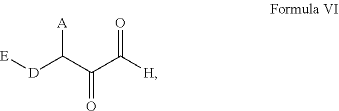

wherein A can be substituted with one or more or H, F, CF₃, CF₂H, CFH₂, CH₃, alkyl group or combinations thereof; D can be a linker; and E can be a be a reactive functional group.

In certain cases, E is a click chemistry moiety selected from alkynes, azides, strained alkynes, dienes, dieneophiles, alkoxyamines, carbonyls, phosphines, hydrazides, thiols, alkenes, and diazirines. In certain cases, E can be selected from alkynes, azides, strained alkynes, dienes, dieneophiles, alkoxyamines, carbonyls, phosphines, hydrazides, thiols, alkenes, diazirines. E can be a substituted alkyl, heteroalkyl, substituted heteroalkyl, heteroaryl, or substituted heteroalkyl. In some cases, E can be a substituted or unsubstituted phenol, substituted or unsubstituted thiophenol, substituted or unsubstituted aniline, substituted or unsubstituted tetrazole, substituted or unsubstituted tetrazine, substituted or unsubstituted SPh, substituted or unsubstituted diazirine, substituted or unsubstituted diazirane, substituted or unsubstituted benzophenone, substituted or unsubstituted nitrone, substituted or unsubstituted nitrile oxide, substituted or unsubstituted norbornene, substituted or unsubstituted nitrile, substituted or unsubstituted isocyanide, substituted or unsubstituted quadricyclane, substituted or unsubstituted alkyne, substituted or unsubstituted azide, substituted or unsubstituted strained alkyne, substituted or unsubstituted diene, substituted or unsubstituted dienophile, substituted or unsubstituted alkoxyamine, substituted or unsubstituted carbonyl, substituted or unsubstituted phosphine, substituted or unsubstituted hydrazide, substituted or unsubstituted thiol, or substituted or unsubstituted alkene. In certain cases, E is a click chemistry compatible reactive group selected from protected thiol, alkene (including trans-cyclooctene [TCO]) and tetrazine inverse-demand Diels-Alder, tetrazole photoclick reaction, vinyl thioether alkynes, azides, strained alkynes, diazranes, dienes, dieneophiles, alkoxyamines, carbonyls, phosphines, hydrazides, thiols, and alkenes. In certain cases, E can be further coupled to a linker (E can be a linker having a terminal click chemistry moiety).

In certain cases, D is a linker selected from an ester, amide, tetrazine, tetrazole, triazine, triazole, aryl groups, heterocycle, sulfonamide, a substituted or unsubstituted —(CH₂)ₙ— where n is 1-10 with 0, 1, 2, 3, 4, 5, 6, 7, 8, 9, 10 methyl substitutions; —O(CH₂)ₘ— where m is 1-10 with 0, 1, 2, 3, 4, 5, 6, 7, 8, 9, 10 methyl substitutions; —NR⁵ — where R⁵ is H or alkyl such as methyl; —NR⁶CO(CH₂)ⱼ— where j is 1-10 with 0, 1, 2, 3, 4, 5, 6, 7, 8, 9, 10 methyl substitutions and R⁶ is H or alkyl such as methyl; or —O(CH₂)ₖR⁶ — where k is 1-10 with 0, 1, 2, 3, 4, 5, 6, 7, 8, 9, 10 methyl substitutions and R¹¹ is alkyl, substituted alkyl, cycloalkyl, substituted cycloalkyl, heteroalkyl, substituted heteroalkyl, aryl, substituted aryl, heteroaryl, or substituted heteroaryl. In some cases, D can be —N(CH₃)—, —OCH₂—, —N(CH₃)COCH₂—, or a group having the chemical formula of Formula VII.

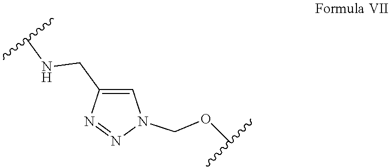 [SMILES: *NCc1cn(CO*)nn1]

In some cases, D can be substituted with a reactive group, e.g., a click chemistry moiety. In some cases, D can be a direct bond between E and the carbon atom binding A. In certain cases, D can be a substituent that modulates the stability of the product formed, selected from alkoxy groups, ethers, carbonyls, aryl groups, electron withdrawing groups (e.g, nitro-, trifluoromethyl-, cyano groups, trimethylsilyl-, esters—either as stand-alone substituents or substituted aryl groups), electron donating groups (e.g, alkyl groups, thiols, amines, aziridines, oxiranes, alkenes—either as stand-alone substituents or substituted aryl groups), electrophilic or nucleophilic centers (e.g, aldehydes, ketones, anhydrides, imines, nitriles, alkenes, alkynes, aryls, heteroaryls), or H-bond acceptors (e.g, ethers, alcohols, carbonyls, amines, thiols, thioethers, sulfonamides, halides).

A is substituted with one or more (mono-substituted, di-substituted, etc.) of H, F, CF₃, CF₂H, CFH₂, CH₃, alkyl group, or combinations thereof. In certain cases,

A can be mono- or di-substituted with a linker. In certain cases, A can be mono- or di-substituted with a reactive group, e.g., a click chemistry moiety, therapeutic agent, or binding moiety. In certain cases, the azide moiety is further coupled to an agent or binding moiety. In certain cases, the agent or binding moiety binds directly or indirectly to a target (protein or nucleic acid) in vivo, ex vivo or in vitro. In certain cases, the agent or binding moiety binds directly or indirectly to a target (protein or nucleic acid) in vivo.

In some cases, reactive groups can be activated by pH changes, oxidation, light, metal or other catalysts. In certain cases, E can contain a detectable label including, but not limited to: a drug, a toxin, a peptide, a polypeptide, an epitope tag, a member of a specific binding pair, a fluorophore, a solid support, a nucleic acid (DNA/RNA), a lipid, or a carbohydrate. In certain cases, E can contain an affinity group including biotin, ligand, substrate, macromolecule with affinity to another molecule, macromolecule, or surface.

The complex can tether an agent or binding moiety to a nucleic, and as such the kethoxal analog acts a tether between a functional agent and a nucleic in proximity to the functional agent. The kethoxal analog is a tether or bifunctional entity, which can be called a biofunctional moiety. The agent can be a small molecule, oligonucleotide, or the like. In certain cases, the agent, binding moiety, or small molecule binds to a protein or a nucleic acid. In certain cases, the agent is a therapeutic agent. The therapeutic agent can be a small molecule, drug, medicine, pharmaceutical, hormone, antibiotic, protein, gene, nucleic acid growth factor, bioactive material, etc., used for treating, controlling, or preventing diseases or medical conditions. In other cases, the agent or therapeutic agent is a nucleic acid. The nucleic acid can be an inhibitory nucleic acid, for example a siRNA. The kethoxal analog can be a N₃-kethoxal and can be operatively couple to agent or binding agent.

In some cases, a kethoxal analog is a compound of Formula VIII below:

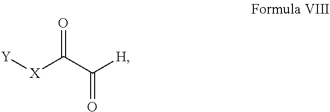

wherein X can be different linkers, such as —CH₂— and —OCH₂—, or polymers, optionally substituted with a reactive group. X can be optional.

Y can be any reactive functional group, including: phenols, thiophenols, anilines, tetrazoles, tetrazines, Sph, diazirines, benzophenones, nitrones, nitrile oxides, norbornenes, nitriles, isocyanides, quadricyclanes, alkynes, azides, strained alkynes, dienes, dienophiles, alkoxyamines, carbonyls, phosphines, hydrazides, thiols, alkenes, etc. Reactive groups can be activated by pH changes, oxidation, light, metal or other catalysts.

Y can also be any detectable label including: a drug, a toxin, a peptide, a polypeptide, an epitope tag, a member of a specific binding pair, a fluorophore, a solid support, a nucleic acid (DNA/RNA), a lipid, or a carbohydrate.

Y can also be any affinity group including biotin, ligand, substrate, macromolecule with affinity to another molecule, macromolecule, or surface.

Certain non-limiting examples of kethoxal analogs are:

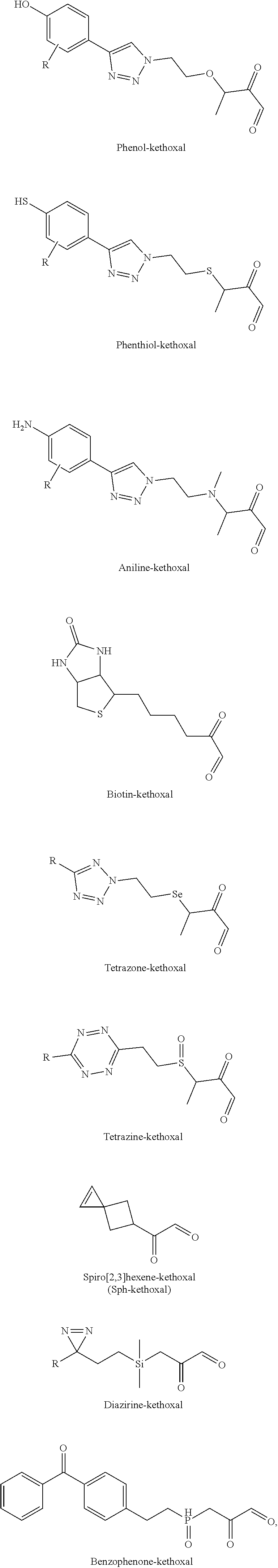

where R can be different substituents.

Kethoxal analogs can be converted into their hydrated form in aqueous solutions according to the following general reaction:

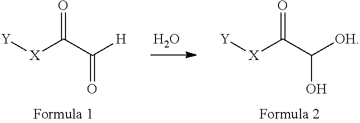

Thus, a kethoxal analog can be in a hydrated form. Additional kethoxal analogs that could be used in the methods disclosed herein are disclosed in WO2020237262, which is incorporated herein by reference in its entirety.

As used herein, the term “biotin moiety” refers to an affinity tag that includes biotin or a biotin analogue such as desthiobiotin, oxybiotin, 2-iminobiotin, diaminobiotin, biotin sulfoxide, biocytin, etc. Biotin moieties bind to streptavidin with an affinity of at least 10⁻⁸ M.

As used herein, the terms “cycloaddition reaction” and “click reaction” are described interchangeably to refer to a 1,3-cycloaddition between an azide and alkyne to form a five membered heterocycle. In some embodiments, the alkyne may be strained (e.g., in a ring such as cyclooctyne) and the cycloaddition reaction may be done in copper free conditions. Dibenzocyclooctyne (DBCO) and difluorooctyne (DIFO) are examples of alkynes that can participate in a copper-free cycloaddition reaction, although other groups are known. See, e.g., Kolb et al. (Drug Discov Today 2003 8: 1128-113), Baskin et al. (Proc. Natl. Acad. Sci. 2007 104: 16793-16797) and Sletten et al. (Accounts of Chemical Research 2011 44: 666-676) for a review of this chemistry.

As used herein, the term “support that binds to biotin” refers to a support (e.g., beads, which may be magnetic) that is linked to streptavidin or avidin, or a functional equivalent thereof. The support can be beads, such as magnetic beads; plate, such as surface modified plate; or particles, such as micro-particles.

The terms “enrich” and “enrichment” refers to a partial purification of analytes that have a certain feature from analytes that do not have the feature. Enrichment typically increases the concentration of the analytes that have the feature by at least 2-fold, at least 5-fold or at least 10-fold relative to the analytes that do not have the feature. After enrichment, at least 10%, at least 20%, at least 50%, at least 80% or at least 90% of the analytes in a sample may have the feature used for enrichment.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) (e.g., of dsRNA duplex of a guide RNA molecule; of a guide RNA base pairing with a target nucleic acid, etc.) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like. “Binding” as used herein (e.g. with reference to an RNA-binding domain of a polypeptide, binding to a target nucleic acid, and the like) refers to a non-covalent interaction between macromolecules (e.g., between a protein and a nucleic acid; between a modified CRISPR/Cas effector polypeptide/guide RNA complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{D}) of less than 10⁻⁶ M, less than 10⁻⁷ M, less than 10⁻⁸ M, less than 10⁻⁸ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{D}.

The term “specific binding” refers to a direct association between two molecules, due to, for example, covalent, electrostatic, hydrophobic, and ionic and/or hydrogen-bond interactions, including interactions such as salt bridges and water bridges.

Binding members of a specific binding pair have binding specificity for one another. A binding member may be naturally derived or wholly or partially synthetically produced. A binding member has an area on its surface, or a cavity, which specifically binds to and is therefore complementary to a particular spatial and polar organization of the other member. Thus, a first binding member specifically binds to a second binding member of a specific binding pair. Examples of specific binding pairs are antigen-antibody, biotin-avidin, hormone-hormone receptor, receptor-ligand, nucleic acids that hybridize with each other, and enzyme-substrate.

Binding members exhibit high affinity and binding specificity for each other. Typically, affinity between the binding members of a specific binding pair is characterized by a Ka (dissociation constant) of 10⁻⁸ M or less, such as 10⁻⁷ M or less, including 10⁻⁸ M or less, e.g., 10⁻⁹ M or less, 10⁻¹⁹ M or less, 10⁻¹¹ M or less, 10⁻¹² M or less, 10⁻¹³ M or less, 10⁻¹⁴ M or less, including 10⁻¹⁵ M or less.

A “test cell,” or “control cell” as used herein, denotes an in vivo or in vitro eukaryotic cell or a cell line.

A “binding site for a guide-RNA” as used herein is a polynucleotide (e.g., DNA such as genomic DNA) that includes a site (“target site” or “target sequence”) targeted by a modified CRISPR/Cas effector polypeptide. The target sequence is the sequence to which the guide sequence of a guide nucleic acid (e.g., guide RNA; e.g., a dual guide RNA or a single-molecule guide RNA) will hybridize. For example, the target site (or target sequence) 5′-GAGCAUAUC-3′ within a target nucleic acid is targeted by (or is bound by, or hybridizes with, or is complementary to) the sequence 5′-GAUAUGCUC-3′. Suitable hybridization conditions include physiological conditions normally present in a cell. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand.”

“Nuclease” and “endonuclease” are used interchangeably herein to mean an enzyme which possesses catalytic activity for nucleic acid cleavage (e.g., ribonuclease activity (ribonucleic acid cleavage), deoxyribonuclease activity (deoxyribonucleic acid cleavage), etc.).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112. In describing and claiming the present invention, certain terminology will be used in accordance with the definitions set out below. It will be appreciated that the definitions provided herein are not intended to be mutually exclusive.

As used herein, the phrases “for example,” “for instance,” “such as,” or “including” are meant to introduce examples that further clarify more general subject matter. These examples are provided only as an aid for understanding the disclosure and are not meant to be limiting in any fashion.

As used herein, the terms “may,” “optional,” “optionally,” or “may optionally” mean that the subsequently described circumstance may or may not occur, so that the description includes instances where the circumstance occurs and instances where it does not.

Definitions of other terms and concepts appear throughout the detailed description.

### DETAILED DESCRIPTION

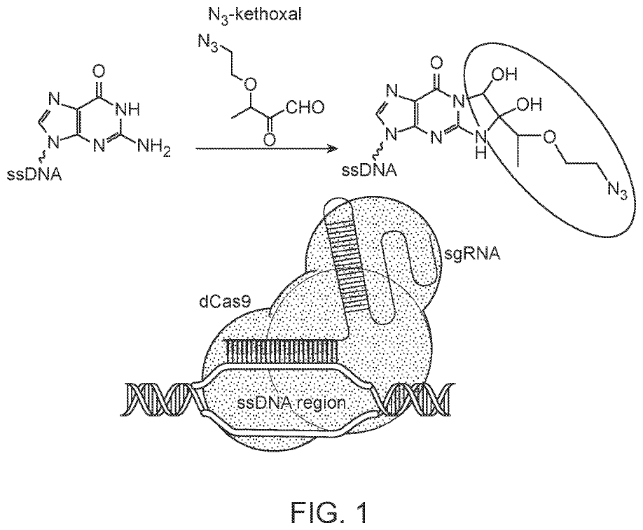

Certain principles of the method are shown in FIG. 1. Kethoxal and its analogs specifically react with guanines in single-stranded DNA, but not double-stranded DNA, under mild (non-denaturing) conditions. A chemoselective group (e.g., an azide) can be added to kethoxal or its analog which, in turn, allows the single-stranded DNA to be labeled with the chemoselective group. The chemoselective group can, in turn, be used to pull out the sequences that were single stranded in the sample. As illustrated in FIG. 1, this principle can be used to investigate the binding sites for RNA-guided endonucleases, e.g., in a genome. As shown, these enzymes create a single stranded sequence during strand invasion. This single stranded sequence can be targeted, isolated and sequenced, thereby providing a way to identify “off-target” binding sites for an RNA-guided endonuclease. The method is high throughput and relative straightforward and is thus believed to be a significant contribution to the art. As shown, a defective RNA-guided endonuclease can be used (i.e., an enzyme that is catalytically inactive but capable of binding to the DNA).

In some embodiments, the method may comprise: (a) contacting the RNA-guided endonuclease with a target double-stranded DNA, thereby producing single stranded DNA (ss-DNA) at the site to which the guide-RNA binds,
(b) reacting the product of step (a) with kethoxal or an analog thereof, modified by a chemoselective group, thereby adding the chemoselective group to any unpaired guanine base within the ssDNA,

The CRISPR system suitable for use in the methods of the present disclosure can be: CRISPR (active Cas9), CRISPRi (CRISPR interference, a catalytically dead Cas9 fused to a transcriptional repressor peptide including KRAB), CRISPRa (CRISPR activation, a catalytically dead Cas9 fused to a transcriptional activator peptide including VPR).

Accordingly, an embodiment of the invention provides a method for isolating DNA fragments that contain a binding site for a guide-RNA of an RNA-guided endonuclease, the method comprising:
(a) contacting the RNA-guided endonuclease with a target double-stranded DNA, thereby producing single stranded DNA (ss-DNA) at the site to which the guide-RNA binds,
(b) reacting the product of step (a) with kethoxal or an analog thereof modified by a chemoselective group, thereby adding the chemoselective group to any unpaired guanine base within the ssDNA,
(c) linking a first binding member of a specific binding pair to the chemoselective group added in step (b),
(d) fragmenting the DNA after step (c) to produce fragmented target DNA, and
(e) enriching for fragments that contain the first binding member of the specific binding pair by binding the product of step (d) to a support that comprises a second binding member of the specific binding pair, which specifically binds to the first binding member of the specific binding pair.

In some cases, linking the first binding member of a specific binding to the chemoselective group can be performed via a cycloaddition reaction.

In some embodiments, the kethoxal modified by a chemoselective group is a compound of Formula IX, below:

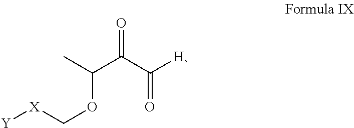

wherein, Y is a chemoselective group selected from alkynes, azides, strained alkynes, dienes, dieneophiles, alkoxyamines, carbonyls, phosphines, hydrazides, thiols, and alkenes; and X is a linker. The linker can be a azide or a C1 to C10 alkyl or polyethylene glycol linker. In some cases, Y is CH₂.

Certain additional compounds that constitute kethoxal modified by a chemoselective group are described in the PCT publication WO2019217533, which is herein incorporated by reference in its entirety.

In certain embodiments, kethoxal or an analog thereof modified by the chemoselective group can be N₃-kethoxal, i.e., the chemoselective group added to the unpaired guanine bases is azido.

The first binding member of the specific binding pair can be a biotin moiety and the second binding member of the specific binding pair can be streptavidin. Isolating from the fragmented DNA, the DNA fragment that contains the binding site for the guide-RNA can be achieved via the specifically binding between a second binding member of the specific binding pair and the first binding member of the specific binding pair conjugated to the DNA fragment. For example, the second member of the specific binding pair can be attached to a bead, for example, a magnetic bead. The DNA fragments containing the first member of the specific binding pair can be immobilized on the beads containing the second member of the specific binding pair. Unbound DNA fragments can be washed away from the beads, thereby isolating only the DNA fragments that contain the first member of the specific binding pair.

Alternatively, the second binding member of the specific binding pair could be attached to a column and the DNA fragments could be flowed through the column thereby capturing the DNA fragments that contain the first member of the specific binding pair.

Any suitable method can be used to capture the DNA fragments that contain the first member of the specific binding pair using the second member of the specific binding pair.

In certain embodiments, the DNA fragments that contain the first member of the specific binding pair is quantified, for example, using a quantitative PCR.

In certain embodiments, the method comprises amplifying the DNA fragments that contain the first binding member of the specific binding pair.

In certain cases, such amplification can be performed without releasing the DNA fragments comprising the first binding member of the specific binding pair from the support used to enrich the DNA fragments. For example, the support that comprises a second binding member of the specific binding pair can be washed to remove non-specifically bound molecules and the support containing the DNA fragments can be used as a template for amplification of the DNA fragments.

In some cases, the target DNA comprising the guanine residue comprising kethoxal or an analog thereof modified by the chemoselective group is purified before linking the chemoselective group to the first binding member of the specific binding pair.

The RNA-guided endonuclease can be an active nuclease or a dead nuclease. Any CRISPR/Cas effector polypeptide is suitable for use in the methods disclosed herein. In some cases, the CRISPR/Cas effector polypeptide used in the methods disclosed herein is a type II CRISPR/Cas effector polypeptide, a type V CRISPR/Cas effector polypeptide, or a type VI CRISPR/Cas effector polypeptide. In some cases, the CRISPR/Cas effector polypeptide used in the methods disclosed herein is a type II CRISPR/Cas effector polypeptide. In some cases, the CRISPR/Cas effector polypeptide used in the methods disclosed herein is a Cas9 polypeptide. In some cases, the CRISPR/Cas effector polypeptide used in the methods disclosed herein is a type V CRISPR/Cas effector polypeptide. In some cases, the CRISPR/Cas effector polypeptide used in the methods disclosed herein is a Cas12a, a Cas12b, a Cas12c, a Cas12d, or a Cas12e polypeptide. In some cases, the CRISPR/Cas effector polypeptide used in the methods disclosed herein is a type VI CRISPR/Cas effector polypeptide. In some cases, the CRISPR/Cas effector polypeptide used in the methods disclosed herein is a Cas13a, a Cas13b, a Cas13c, or a Cas13d polypeptide. In some cases, the CRISPR/Cas effector polypeptide used in the methods disclosed herein is a Cas14a, a Cas14b, or a Cas14c polypeptide. Amino acid sequences of a variety of CRISPR/Cas effector polypeptides are known. Examples of various Cas9 proteins (and Cas9 domain structure) and Cas9 guide RNAs (as well as information regarding requirements related to protospacer adjacent motif (PAM) sequences present in targeted nucleic acids) can be found in the art.

In some cases, a CRISPR/Cas effector polypeptide used in the methods disclosed herein is enzymatically active. In some cases, a CRISPR/Cas effector polypeptide used in the methods disclosed herein exhibits reduced enzymatic activity compared to a wild-type CRISPR/Cas effector polypeptide. In some cases, a CRISPR/Cas effector polypeptide used in the methods disclosed herein is a nickase. In some cases, a CRISPR/Cas effector polypeptide used in the methods disclosed herein is enzymatically inactive (a “dead” CRISPR/Cas effector polypeptide) but retains the ability to bind a target nucleic acid when complexed with a guide nucleic acid.

A guide nucleic acid suitable for inclusion in a system of the present disclosure can include: i) a first segment (referred to herein as a “targeting segment”); and ii) a second segment (referred to herein as a “protein-binding segment”). By “segment” it is meant a segment/section/region of a molecule, e.g., a contiguous stretch of nucleotides in a nucleic acid molecule. A segment can also mean a region/section of a complex such that a segment may comprise regions of more than one molecule. The “targeting segment” is also referred to herein as a “variable region” of a guide RNA. The “protein-binding segment” is also referred to herein as a “constant region” of a guide RNA. The first segment (targeting segment) of a guide RNA includes a nucleotide sequence (a guide sequence) that is complementary to (and therefore hybridizes with) a specific sequence (a target site) within a target nucleic acid (e.g., a target ssRNA, a target ssDNA, the complementary strand of a double stranded target DNA, etc.). The protein-binding segment (or “protein-binding sequence”) interacts with (binds to) a CRISPR/Cas effector polypeptide. The protein-binding segment of a guide RNA includes two complementary stretches of nucleotides that hybridize to one another to form a double stranded RNA duplex (dsRNA duplex). Site-specific binding and/or cleavage of a target nucleic acid (e.g., genomic DNA) can occur at locations (e.g., target sequence of a target locus) determined by base-pairing complementarity between the guide RNA (the guide sequence of the guide RNA) and the target nucleic acid.

A guide RNA and a CRISPR/Cas effector polypeptide form a complex (e.g., bind via non-covalent interactions). The guide RNA provides target specificity to the complex by including a targeting segment, which includes a guide sequence (a nucleotide sequence that is complementary to a sequence of a target nucleic acid). The CRISPR/Cas effector polypeptide of the complex provides the site-specific activity (e.g., cleavage activity or an activity provided by the CRISPR/Cas effector polypeptide when the CRISPR/Cas effector polypeptide is a CRISPR/Cas effector polypeptide fusion polypeptide, i.e., has a fusion partner). In other words, the CRISPR/Cas effector polypeptide is guided to a target nucleic acid sequence (e.g. a target sequence in a chromosomal nucleic acid, e.g., a chromosome; a target sequence in an extrachromosomal nucleic acid, e.g. an episomal nucleic acid, a minicircle, an ssRNA, an ssDNA, etc.; a target sequence in a mitochondrial nucleic acid; a target sequence in a chloroplast nucleic acid; a target sequence in a plasmid; a target sequence in a viral nucleic acid; etc.) by virtue of its association with the guide RNA.

The “guide sequence” also referred to as the “targeting sequence” of a guide RNA can be modified so that the guide RNA can target a CRISPR/Cas effector polypeptide to any desired sequence of any desired target nucleic acid, with the exception that the protospacer adjacent motif (PAM) sequence can be considered. Thus, for example, a guide RNA can have a targeting segment with a sequence (a guide sequence) that has complementarity with (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some cases, a guide RNA includes two separate nucleic acid molecules: an “activator” and a “targeter” and is referred to herein as a “dual guide RNA”, a “double-molecule guide RNA”, or a “two-molecule guide RNA” a “dual guide RNA”, or a “dgRNA.” In some embodiments, the activator and targeter are covalently linked to one another (e.g., via intervening nucleotides) and the guide RNA is referred to as a “single guide RNA”, a “Cas9 single guide RNA”, a “single-molecule Cas9 guide RNA,” or a “one-molecule Cas9 guide RNA”, or simply “sg RNA.”

The target DNA can be a genomic nucleic acid, a mitochondrial nucleic acid; a chloroplast nucleic acid; a plasmid; or a viral nucleic acid. The target DNA can be isolated from a cell or can be within an intact cell.

In certain cases, the DNA fragment that contains the binding site for the guide-RNA as isolated according to the methods disclosed herein is sequenced, for example, via a next generation sequencing (NGS) method. The NGS method can be any convenient sequencing protocol, such as but not limited to paired-end sequencing, ion-proton sequencing, pyrosequencing, nanopore sequencing. A person of ordinary skill in the art can readily identify and use appropriate sequencing methods to determine the sequences of the isolated DNA fragment.

Sequencing the DNA fragment that contains the binding site for the guide-RNA can be used to identify off-target binding sites for the RNA-guided endonuclease. For example, the sequence of the DNA fragment that contains the binding site for the guide-RNA can be compared with a desired or expected binding site for the guide-RNA. If the sequences are identical, then the identified binding site is genuine “on-target” binding site for the guide-RNA. However, if the sequences are not identical, then the identified binding site is not genuine and, hence, is “off-target” binding site for the guide-RNA. Accordingly, the methods disclosed herein could be used to identify whether a selected guide-RNA is appropriate for use, for example, has no “off-target” binding and is specific only to the “on-target” binding site.

The RNA-guided endonuclease can be contacted with a target DNA that is outside a cell or inside a cell.

When the target DNA is inside a cell, the RNA-guided endonuclease, for example, Cas9 or dCas9 and the guide-RNA, are transfected into the cells to contact the RNA-guided endonuclease with the genomic DNA of the cell. An example of such transfection method is disclosed in the “Materials and Methods” below under “In vivo CasKAS.”

Living cells contain substantial ssDNA due to active transcription and other processes. Therefore, N₃-kethoxal can bind to the guanine residues in such ssDNA and produce a binding member conjugated DNA fragment. To distinguish such DNA fragments from guide-RNA mediated ssDNA fragments, a control cell is used. The control cell is transfected with an RNA-guided endonuclease that is without the guide-RNA. Thus, identifying DNA fragments containing the ssDNA in such control cell provides a map of background endogenous ssDNA profile. The background profile can then be compared to DNA fragments obtained from a test cell, which is transfected with RNA-guided endonuclease that contains the guide-RNA.

Accordingly, certain embodiments of the invention comprise:
(g) contacting an RNA-guided endonuclease without the guide RNA with the target double-stranded DNA inside a control cell, thereby producing ss-DNA at the site to which the guide-RNA binds,
(h) reacting the product of step (g) with kethoxal or an analog thereof modified by a chemoselective group, thereby adding the chemoselective group to any unpaired guanine base within the ssDNA,
(i) linking a first binding member of a second specific binding pair to the chemoselective group added in step (h),
(j) fragmenting the DNA after step (i) to produce fragmented target DNA from the control cell,
(k) enriching for fragments that contain the first binding member of the second specific binding pair by binding the product of step (j) to a support that comprises a second binding member of the second specific binding pair, which specifically binds to the first binding member of the second specific binding pair.

In some cases, linking the first binding member of the second specific binding pair to the chemoselective group can be done via a cycloaddition reaction.

Like the methods described above, kethoxal or an analog thereof modified by a chemoselective group can be N₃-kethoxal, i.e., the chemoselective group added to the unpaired guanine bases is azido.

Also, the first binding member of the second specific binding pair is a biotin moiety and the second binding member of the second specific binding pair is streptavidin.

Isolating from the fragmented DNA, the DNA fragment that contains the binding site for the guide-RNA can be achieved via the specifically binding between a second binding member of the second specific binding pair and the first binding member of the second specific binding pair conjugated to the DNA fragment. For example, the second member of the second specific binding pair can be attached to a bead, for example, a magnetic bead. The DNA fragments containing the first member of the second specific binding pair can be immobilized on the beads containing the second member of the second specific binding pair. Unbound DNA fragments can be washed away from the beads, thereby isolating only the DNA fragments that contain the first member of the second specific binding pair.

Alternatively, the second binding member of the second specific binding pair could be attached to a column and the DNA fragments could be flowed through the column thereby capturing the DNA fragments that contain the first member of the second specific binding pair.

Any suitable method can be used to capture the DNA fragments that contain the first member of the specific second binding pair using the second member of the second specific binding pair.

In certain embodiments, the DNA fragments that contain the first member of the specific second binding pair is quantified, for example, using a quantitative PCR.

In certain embodiments, the method comprises amplifying the DNA fragments that contain the first binding member of the second specific binding pair.

In certain cases, such amplification can be performed without releasing the DNA fragments comprising the first binding member of the second specific binding pair from the support used to enrich the DNA fragments. For example, the support that comprises a second binding member of the second specific binding pair can be washed to remove non-specifically bound molecules and the support containing the DNA fragments can be used as a template for amplification of the DNA fragments.

The amplified DNA fragments can be sequenced for example, via a next generation sequencing method.

The sequences of the DNA fragments enriched from the control cell could be used to determine a map of background endogenous ssDNA profiles. Such map can be compared to the sequence of the DNA fragments enriched from a test cell, i.e., a cell contacted with a guide-RNA containing endonuclease, can be used to identify the binding sites for a guide-RNA of an RNA-guided endonuclease.

### KITS

Also provided are kits having one or more components and/or reagents and/or devices, where applicable, for practicing one or more of the above-described methods. The subject kits may vary greatly. Kits of interest include those having one or more reagents mentioned herein, and associated devices where applicable, with respect to the steps of: (a) contacting the RNA-guided endonuclease with a target double-stranded DNA, (b) reacting the product of step (a) with kethoxal or an analog thereof, modified by a chemoselective group, (c) linking a first binding member of a specific binding pair to the chemoselective group added in step (b), (d) fragmenting the DNA after step (c) to produce fragmented target DNA, and (e) enriching for fragments that contain the first binding member of the specific binding pair.

Kits may include certain combinations of components in a single reaction vessel. Kits may include different components in different vessels.

In some cases, a kit comprises: 1) an RNA-guided endonuclease, 2) kethoxal or an analog thereof, modified by a chemoselective group, and, optionally, 3) a first binding member of a specific binding pair having a functional group reactive to the chemoselective group of kethoxal or analog thereof.

The kit can further contain a support that comprises a second binding member of the second specific binding pair, which specifically binds to the first binding member of the second specific binding pair. The support can be beads, such as magnetic beads; plate, such as surface modified plate; or particles, such as micro-particles.

In some cases, the first binding member of the specific binding pair is biotin moiety and the second binding member of the specific binding pair is streptavidin.

A person of ordinary skill in the art can readily design a kit according to the details of the methods disclosed above and such embodiments are within the purview of the invention.

### UTILITY

The present method is relatively rapid and inexpensive. For example, in some case it can be carried out within ˜8 hours for an in vitro experiment and less than 24 hours for an in vivo experiment. Additional time for sequencing and analysis on the order of 16 hours is sufficient. It can be implemented using relatively straightforward molecular biology procedures and can thus be readily adopted outside labs with high-level of technological expertise. The method does not require resequencing of a whole genome as it actively enriches for off-target sites, thus it is also inexpensive. The method can be applied to all different types of DNA-targeting CRISPR proteins, unlike all other methods which only map on type of CRISPR. CasKAS can profile both CRISPR occupancy and CRISPR cleavage. As it measures physical occupancy and not DNA editing outcome, it can be applied to primary non-diving cells.

The methods described in this disclosure find use in a variety of applications. Applications of interest include but are not limited to: research applications and therapeutic applications. Methods of the invention find use in a variety of different applications including any convenient application where identifying off-target effects of CRISPR-mediated genomic editing is desired.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

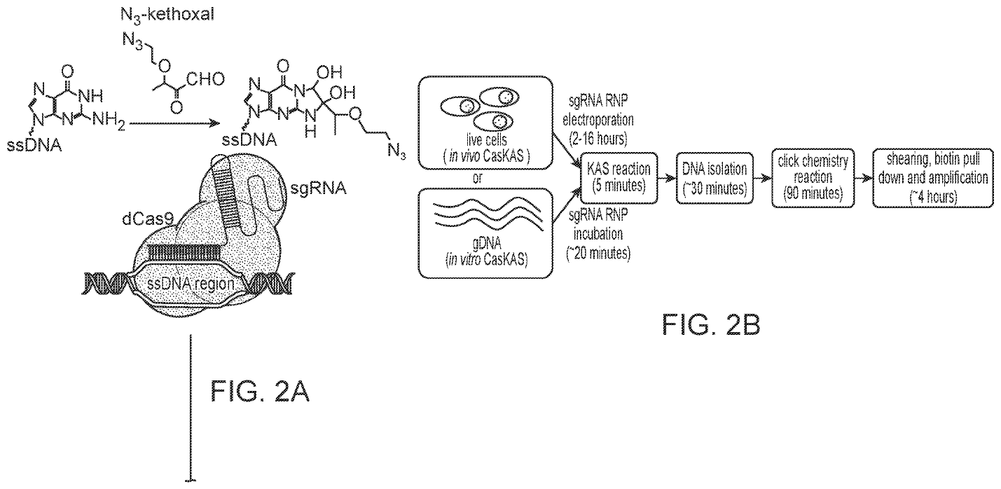

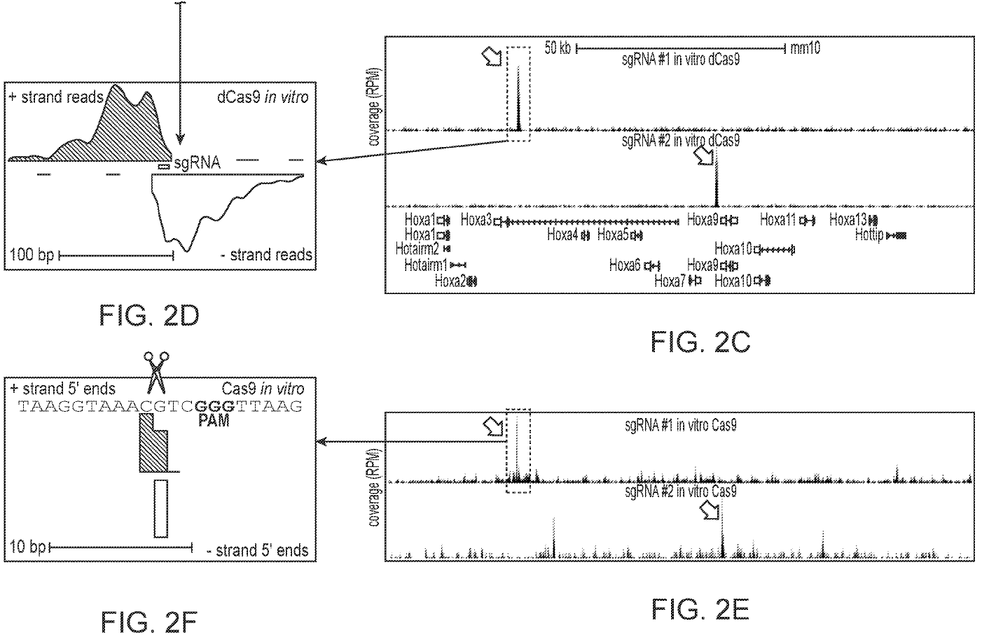

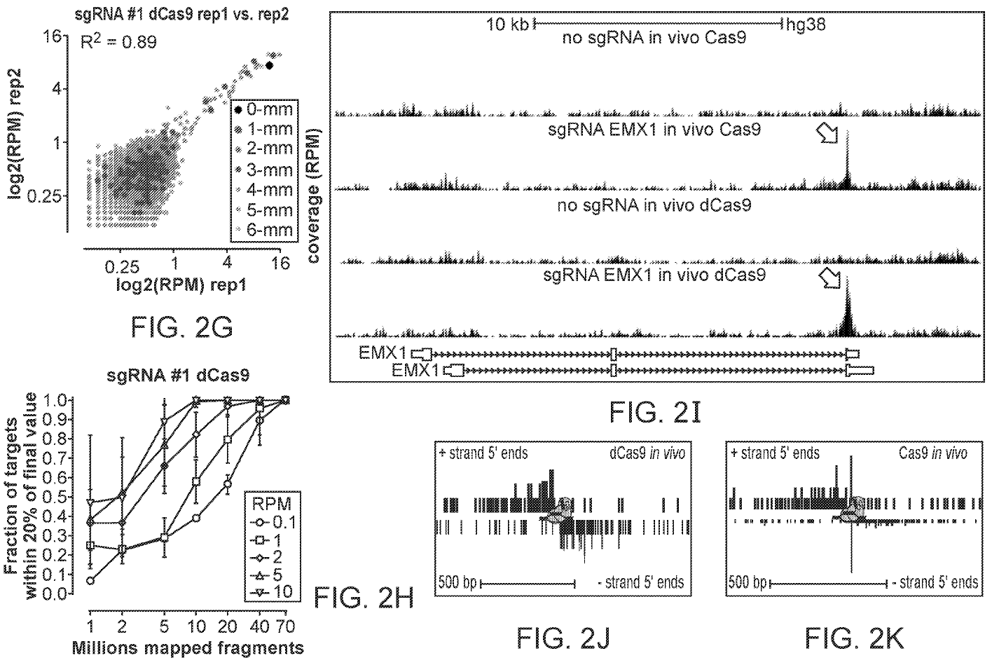

The method disclosed herein, which may be referred to as kethoxal-assisted ssDNA sequencing or “KAS-seq” for short can be used to identify ssDNA structures generated by CRISPR protein binding to DNA (FIG. 2A-B). KAS-seq method is based on the specific covalent labeling of unpaired guanine bases with N₃-kethoxal, generating an adduct to which biotin can then be added using click chemistry. After shearing, biotinylated DNA, corresponding to regions containing ssDNA structure, can be specifically enriched for and sequenced. Specifically, when a Cas9-sgRNA ribonucleoprotein (RNP) is engaged with its target site, the sgRNA invades the DNA double helix, forming a ssDNA structure on the other strand (FIG. 2A). Thus, mapping ssDNA-containing regions would be a sensitive biochemical signal of productive Cas9 binding.

The method is exemplified using N₃-kethoxal. However, analogs of kethoxal and other chemoselective groups could be used.

### Materials and Methods

### Guide RNA Sequences

Guide RNAs were obtained from IDT (“sgRNA #1” and “sgRNA #2”) or from Synthego (all others). The following sgRNA sequences were used in this study:

1. ″sgRNA #1″:
(SEQ ID NO: 1)
GCTTAATTAAGGTAAACGTC
2. ″sgRNA #2″:
(SEQ ID NO: 2)
CCAACCTGGCGGCTCGTTGG
3. ″EMX1 Tsai″:
(SEQ ID NO: 3)
GAGTCCGAGCAGAAGAAGAA
4. ″VEGFA-site1″:
(SEQ ID NO: 4)
GGGTGGGGGGAGTTTGCTCC
5. ″Nanog-sg2″:
(SEQ ID NO: 5)
GATCTCTAGTGGGAAGTTTC
6. ″Nanog-sg3″:
(SEQ ID NO: 6)
GTCTGTAGAAAGAATGGAAG

Guide RNAs were dissolved to a concentration of 100 μM using nuclease-free 1×TE buffer and stored at —20° C.

### In Vitro CasKAS

In vitro CasKAS experiments were executed as follows.

First, 1 μL of each synthetic sgRNA were incubated at room temperature with 1 μL of recombinant purified dCas9 (MCLab dCAS9B-200) for 20 minutes. The RNP was then incubated with 1 μg of gDNA at 37° C. for 10 minutes.

The KAS reaction was then carried out by adding 1 μL of 500 mM N₃-kethoxal (ApeXBio A8793). DNA was immediately purified using the MinElute PCR Purification Kit (Qiagen 28006), and eluted in 87.5 or 175 μL 25 mM K₃BO₃.

### In Vivo CasKAS

For in vivo CasKAS experiments, HEK293T cells were seeded at 400,000 cells/well into a 6-well plate the day before RNP transfection. Media was exchanged 2 hours before transfection. For each well, 6,250 ng of Cas9 (MCLAB CAS9-200) or dCas9 (MCLAB dCAS9B-200) and 1,200 ng sgRNA was complexed with CRISPRMAX reagent in Opti-MEM following manufacturer's protocol. After incubation at room temperature for 15 minutes, the RNP solution was directly added to each well and gently mixed. The cells were incubated with the RNP complex for 14 hours at 37° C. To harvest and perform kethoxal labeling, media was removed and room temperature 1×PBS was used to wash the cells. Cells were then dissociated with trypsin, trypsin was quenched with media, cells were pelleted at room temperature, and then resuspended in 100 μL of media supplemented with 5 M N₃-kethoxal. Cells were incubated for 10 minutes at 37° C. with shaking at 500 rpm in a Thermomixer. Cells were then pelleted by centrifuging at 500 g for 5 minutes at 4° C. Genomic DNA was then extracted using the Monarch gDNA Purification Kit (NEB T3010S) following the standard protocol but with elution using 85 μL 25 mM K₃BO₃ at pH 7.0.

### Click Reaction, Biotin Pull Down and Library Generation

The click reaction was carried out by combining 175 μL purified and sheared DNA, 5 μL 20 mM DBCO-PEG4-biotin (DMSO solution, Sigma 760749), and 20 μL 10×PBS in a final volume of 200 μL or 87.5 μL purified and sheared DNA, 2.5 μL 20 mM DBCO-PEG4-biotin (DMSO solution, Sigma 760749), and 10 μL 10×PBS in a final volume of 100 μL. The reaction was incubated at 37° C. for 90 minutes. DNA was purified using AMPure XP beads (50 μL for a 100 μL reaction or 100 μL for a 200 μL reaction), beads were washed on a magnetic stand twice with 80% EtOH, and eluted in 130 μL 25 mM K₃BO₃.

Purified DNA was then sheared on a Covaris E220 instrument down to ˜150-400 bp size.

For streptavidin pulldown of biotin-labeled DNA, 10 μL of 10 mg/mL Dynabeads MyOne Streptavidin T1 beads (Life Technologies, 65602) were separated on a magnetic stand, then washed with 300 μL of 1×TWB (Tween Washing Buffer; 5 mM Tris-HCl pH 7.5; 0.5 mM EDTA; 1 M NaCl; 0.05% Tween 20). The beads were resuspended in 300 μL of 2×Binding Buffer (10 mM Tris-HCl pH 7.5, 1 mM EDTA; 2 M NaCl), the sonicated DNA was added (diluted to a final volume of 300 μL if necessary), and the beads were incubated for 5 minutes at room temperature on a rotator. After separation on a magnetic stand, the beads were washed with 300 μL of 1×TWB, and heated at 55° C. in a Thermomixer with shaking for 2 minutes. After removal of the supernatant on a magnetic stand, the TWB wash and 55° C. incubation were repeated.

Final libraries were prepared on beads using the NEBNext Ultra II DNA Library Prep Kit (NEB, #E7645) as follows. End repair was carried out by resuspending beads in 50 μL 1×EB buffer, and adding 3 μL NEB Ultra End Repair Enzyme and 7 μL NEB Ultra End Repair Enzyme, followed by incubation at 20° C. for 30 minutes (in a Thermomixer, with shaking at 1,000 rpm) and then at 65° C. for 30 minutes.

Adapters were ligated to DNA fragments by adding 30 μL Blunt Ligation mix, 1 μL Ligation Enhancer and 2.5 μL NEB Adapter, incubating at 20° C. for 20 minutes, adding 3 μL USER enzyme, and incubating at 37° C. for 15 minutes (in a Thermomixer, with shaking at 1,000 rpm).

Beads were then separated on a magnetic stand, and washed with 300 μL TWB for 2 minutes at 55° C., 1000 rpm in a Thermomixer. After separation on a magnetic stand, beads were washed in 100 μL 0.1×TE buffer, then resuspended in 15 μL 0.1×TE buffer, and heated at 98° C. for 10 minutes.

For PCR, 5 μL of each of the i5 and i7 NEB Next sequencing adapters were added together with 25 μL 2×NEB Ultra PCR Mater Mix. PCR was carried out with a 98° C. incubation for 30 seconds and 12 cycles of 98° C. for 10 seconds, 65° C. for 30 seconds, and 72° C. for 1 minute, followed by incubation at 72° C. for 5 minutes.

Beads were separated on a magnetic stand, and the supernatant was cleaned up using 1.8×AMPure XP beads.

Libraries were sequenced in a paired-end format on a Illumina NextSeq instrument using NextSeq 500/550 high output kits (2×36 cycles).

### Data Processing

Demultipexed fastq files were mapped to the hg38 assembly of the human genome or the mm10 version of the mouse genome as 2×36-mers using Bowtie with the following settings: -v 2 -k 2 -m 1 —best —strata -X 1000. Duplicate reads were removed using picard-tools (version 1.99).

Browser tracks generation, fragment length estimation, TSS enrichment calculations, and other analyses were carried out using custom-written Python scripts (see world-wide-website: github.com/georgimarinov/GeorgiScripts). The refSeq set of annotations were used for evaluation of enrichment around TSSs.

### Peak Calling

Peak calling on in vitro binding datasets was carried out using version 2.1.0 of MACS2²⁹ with default settings. Peaks were then compared against the ENCODE set of “blacklisted” regions³⁰ to filter out likely artifacts.

### Sequence Analysis

Guide RNA off-target predictions were obtained from Cas-OFFinder. Multiple sequence alignments of sgRNA sequences and their off-targets were generated using MUSCLE and visualized using JalView.

### Quantification Cutting Score Calculation

The Cas9 cutting C-score was calculated as follows. First, basepair-level Read-Per-Million (RPM) profiles for mapped read 5′ ends were generated separately for the forward and reverse strands. Then the C-score was calculated by multiply the forward and reverse strand profiles (summed over a running window of 3 bp):

$$\begin{matrix}\begin{matrix}j = i + 1 & j = i + 1\end{matrix} \\ C - score c , i = X RPMc , j + \times X RPMc , j - \\ \begin{matrix}j = i - 1 & j = i - 1\end{matrix}\end{matrix} \qquad ( 1 )$$

Where c and i indicate the coordinates by chromosome and position.

The following example(s) is/are offered by way of illustration and not by way of limitation.

### Results

To determine if KAS-seq can be used to map regions of ssDNA generated by Cas9 binding, an initial in vitro experiment was conducted using mouse genomic DNA (gDNA), purified dCas9 and two sgRNAs targeting the Hoxa locus. Strong and highly specific peaks at the expected target sites for each sgRNA (FIG. 1C) were observed. Detailed examination of dCas9 CasKAS profiles around the predicted sgRNA target sites revealed strand coverage asymmetry patterns similar to those observed for ChIP-seq around transcription factor binding sites (FIG. 2D), indicating that enrichment derives from the sgRNA target site itself and confirming the utility of N₃-kethoxal for mapping dCas9 occupancy sites. The assays disclosed herein are referred to as “CasKAS.”

It was hypothesized that CasKAS should also capture active Cas9 complexed with DNA, as the enzyme is thought to remain associated with DNA for some time after cleavage. Cas9 CasKAS experiments were carried out with the same sgRNAs and again enrichment was observed at the expected on-target sites (FIG. 2E).

Remarkably, examination of Cas9 CasKAS read profiles around the on-target site showed that the 5′ ends of reads are precisely positioned around the expected cut site, with one cut position on one strand and two to three such positions on the other (FIG. 1F), consistent with the previously known patterns of Cas9 cleavage. CasKAS therefore provides target specificity profiles for both active and catalytically dead Cas9 versions.

In vitro CasKAS data is highly reproducible between replicates (FIG. 2G), and a modest sequencing depth of between 10 and 20 million mapped reads is generally sufficient to capture off-target specificity profiles (FIG. 2H). Similar results were observed with two mouse sgRNAs targeting the Nanog locus (data not shown) and with two human sgRNA (“EMX1” and “VEGFA”; data not shown). No enrichment was found using components of the RNP in isolation—sgRNAs, dCas9 or Cas9 (not shown).

Next, the application of CasKAS was tested in vivo. Living cells contain substantial ssDNA due to active transcription and other processes, so the in vivo CasKAS signal is a mixture of signals from ssDNA associated with the Cas9 RNP and endogenous processes that generate ssDNA. KAS-seq experiments were conducted using both dCas9 and Cas9 in HEK293 cells transfected with EMX1 or VEGFA RNPs, as well as negative, no-guide controls, which provide a map of background endogenous ssDNA profiles. At the EMX1 gene, which is not active in HEK293 cells, strong peaks were observed at the expected target site (FIG. 21), an asymmetric read profile around it for dCas9 (FIG. 2J), and a substantial degree of 5′ end clustering at the cut site, similar to what is observed in vitro for active Cas9 (FIG. 2G). The VEGFA gene is active in HEK293 cells, but the dCas9/Cas9 CasKAS signal is still readily identifiable as an addition to the endogenous ssDNA enrichment pattern (data not shown). These results demonstrate the utility of CasKAS for profiling CRISPR specificity both in vitro and in vivo

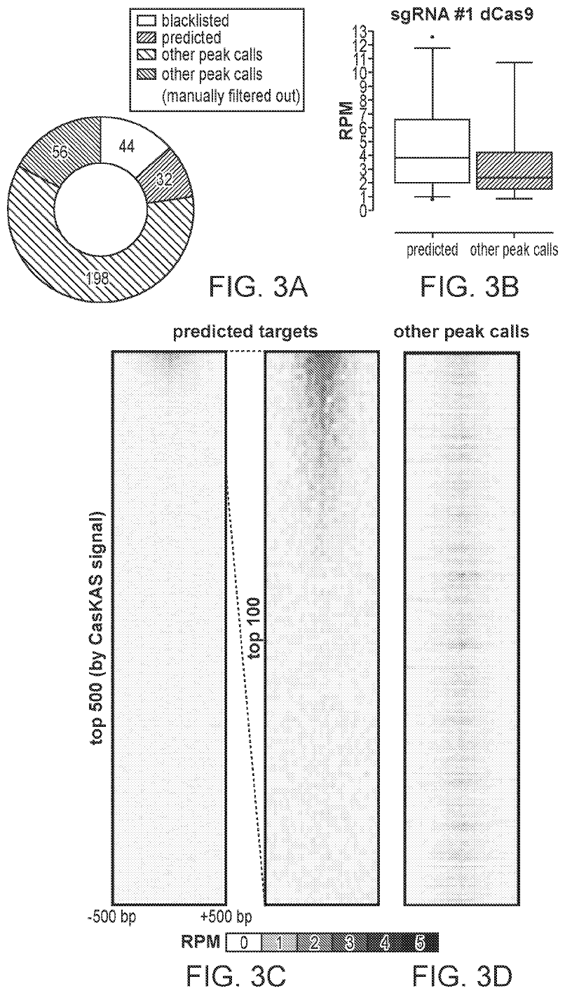

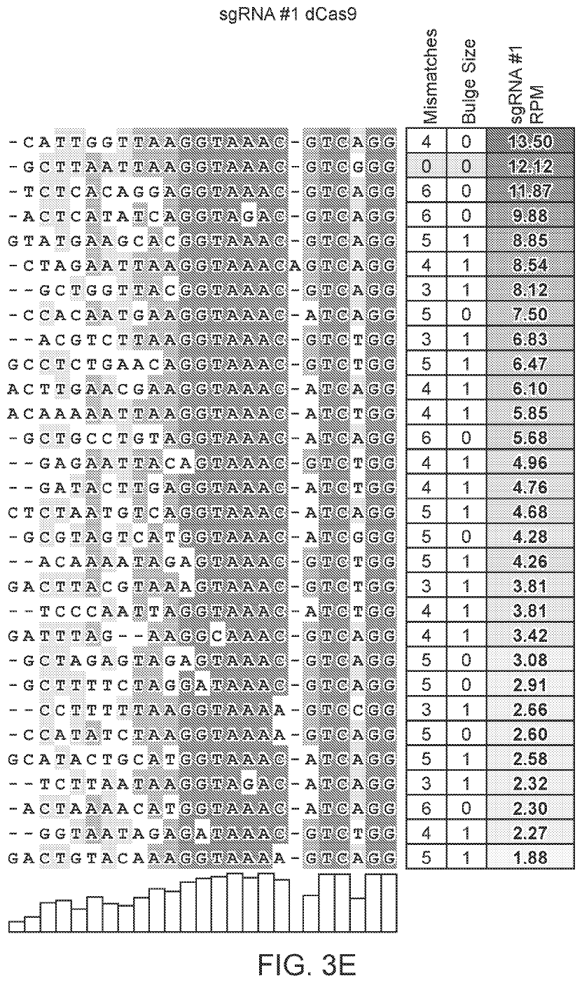

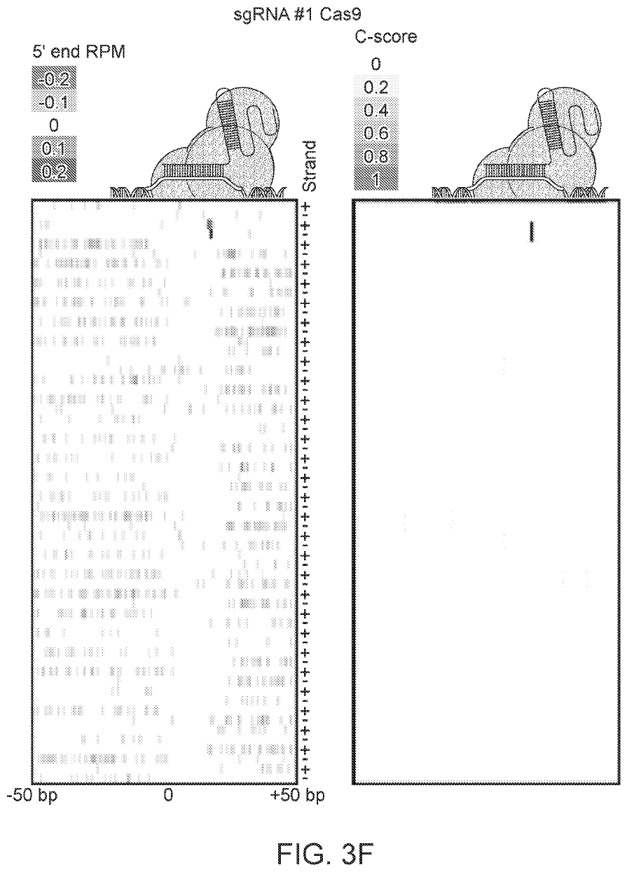

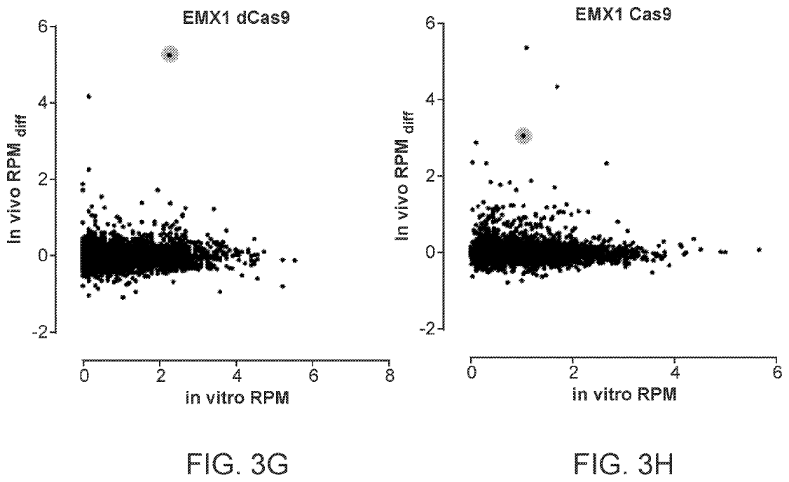

The genome-wide specificity of sgRNAs was next examined as measured by CasKAS. The mouse sgRNA #1 was selected as it displayed a substantial number of off-targets yet that number was also sufficiently small for all of them to be examined directly. First, peaks were called de novo without relying on off-target prediction algorithms, then the resulting peak set was manually curated (FIG. 3A).

Remarkably, while 32 peaks were found at predicted off-target sites, 192 (i.e. ˜6× as many) additional manually curated peaks were also found; while these peaks exhibit generally lower CasKAS signal (FIG. 3B), they all appear to be genuine sites of occupancy as they display proper peak shape characteristics. Most of the predicted (in total ˜7,500) off-target sites for this sgRNA do not show substantial occupancy by dCas9 CasKAS (FIG. 3C-D).

Sequence comparison of the occupied predicted off-target sites allowed evaluating determinants of Cas9 specificity (FIG. 3E). Consistent with previous reports, the PAM-distal region is much less sequence-constrained than the PAMproximal one. A similar pattern was observed with the other profiled sgRNAs, in both mouse and human (data not shown).

When analyzing peaks not associated with predicted off-target sites other telling patterns were observed — at numerous sites with strong dCas9 CasKAS signal, a large number of mismatches to the sgRNA sequence were observed as well as “bulge” regions wherein indels were observed in the target sequence. These mismatches and bulges were in general much larger than what is considered permissible by off-target prediction algorithms; the lack of consideration of potential target sequences with large numbers of mismatches or substantial insertions likely explains the much larger number of such sites compared to the set of occupied predicted off-targets.

A simple metric was devised for evaluating the degree of read clustering at cut sites (a “C-score”; see Methods for details), and was used to estimate the degree of cutting by Cas9. Strikingly, while the on-target site exhibits the second highest dCas9 CasKAS signal, and even though all off-target sites show binding by CasKAS, only the on-target site displays strong cutting activity (FIG. 3F). The behavior of other sgRNAs varies (data not shown), with some showing multiple cut sites. Thus combining dCas9 and Cas9 CasKAS (or even Cas9 CasKAS alone) provides a powerful tool for detecting both binding specificity and the promiscuity of catalytic activity for arbitrary sgRNAs.

Finally, in vitro and in vivo CasKAS profiles were compared (FIG. 3G-H). Many fewer strongly enriched sites were observed in vivo datasets than in vitro, with the on-target site being either the top (for dCas9) or among the top (for Cas9) sites in vivo. A potential explanation for this difference is the previously reported impediment of Cas9/dCas9 binding to DNA by the presence of nucleosomes; this inhibitory effect need not be complete to generate the observed patterns as CasKAS measures the physical occupancy of DNA by CRISPR proteins at the moment of harvesting cells In conclusion, a new, simple and robust method is presented for mapping the specificity of active and catalytically dead versions of CRISPR enzymes. CasKAS has numerous advantages over existing tools while also opening up new possibilities for studying CRISPR biology. CasKAS requires no specialized molecular biology protocols, takes just a few hours in vitro (and a similar amount of time after harvesting cells in vivo), and is inexpensive as it actively and strongly enriches for off-targets. It measures strand invasion by CRISPR rather than association with DNA, a biochemically more specific event. CasKAS peaks were compared de novo to those generated by other means, and while large sets of peaks were found to be unique to each method, those found only by CasKAS contained much higher fractions of predicted off-target sites than those unique to other methods. CasKAS can be used to profile the specificity of all types of DNA-targeting CRISPR proteins as it does not rely on measuring DNA cleavage or modification. CasKAS may be applied in primary cells as what is measured is physical association with DNA and not the outcome of CRISPR activity that may only be detectable after cell division. A limitation of CasKAS is the requirement that a G nucleotide is present within the sgRNA sequence, as without it there would be no kethoxal labeling; however, only a small fraction (≤5%) of sgRNAs in the human genome lack any Gs. Another minor limitation of the current in vitro protocol is that labeling is carried out on high molecular weight (HMW) DNA and samples must sheared serially. Pre-sheared and end-repaired DNA was used (to minimize kethoxal labeling of Gs on sticky ends generated by sonication), with comparable results to using HMW DNA; further optimization should allow the parallel high-throughput plate-based profiling of the specificity of very large numbers of sgRNAs.

In addition to being highly valuable for off-target profiling in vitro and in previously difficult to assay settings such as primary cells, CasKAS can be expected to provide fruitful insights into the mechanisms and dynamics of in vivo CRISPR action (taking advantage of finely controllable CRISPR systems such as vfCRISPR), and the influence of transcriptional, regulatory, and epigenetic and other functional genomic contexts on CRISPR activity.

We claim:

1. A method for isolating DNA fragments that contain a binding site for a guide-RNA of an RNA-guided endonuclease, the method comprising:
(a) contacting the RNA-guided endonuclease with a target double-stranded DNA, thereby producing single stranded DNA (ss-DNA) at the site to which the guide-RNA binds,
(b) reacting the product of step (a) with kethoxal or an analog thereof, modified by a chemoselective group, thereby adding the chemoselective group to any unpaired guanine base within the ssDNA,
(c) linking a first binding member of a specific binding pair to the chemoselective group added in step (b),
(d) fragmenting the DNA after step (c) to produce fragmented target DNA, and
(e) enriching for fragments that contain the first binding member of the specific binding pair by binding the product of step (d) to a support that comprises a second binding member of the specific binding pair, which specifically binds to the first binding member of the specific binding pair.

2. The method of claim 1, wherein the kethoxal or an analog thereof, modified by the chemoselective group is N₃-kethoxal, and wherein the chemoselective group added to the unpaired guanine bases is azido.

3. The method of claim 1, wherein the first binding member of the specific binding pair is a biotin moiety and the second binding member of the specific binding pair is streptavidin.

4. The method of any of preceding claims, further comprising:
(f) amplifying the enriched DNA from step (e).

5. The method of claim 4, wherein the method does not comprise releasing the DNA fragments comprising the first binding member of the specific binding pair from the support after step (e), prior to step (f).

6. The method of claim 5, wherein step (f) comprises:
i. washing the support of step (e) after the DNA fragments comprising the first binding member of the specific binding pair have bound to the support; and then
ii. setting up an amplification reaction containing the support, without releasing from the support the DNA fragments comprising the first binding member of the specific binding pair.

7. The method of any of the preceding claims, comprising purifying the target DNA comprising the guanine residue comprising kethoxal or an analog thereof modified by the chemoselective group before linking the chemoselective group to the first binding member of the specific binding pair.

8. The method of any of the preceding claims, wherein the target DNA is a genomic DNA.

9. The method of any of preceding claims, wherein the RNA-guided endonuclease is an active nuclease.

10. The method of any of preceding claims, wherein the RNA-guided endonuclease is a dead nuclease.

11. The method of any of the preceding claims, further comprising sequencing the DNA fragment that contains the binding site for the guide-RNA.

12. The method of claim 11, wherein the sequencing is performed via a next generation sequencing method.

13. The method of claim 11 or 12, further comprising identifying an off-target binding site for the RNA-guided endonuclease by comparing the sequence of the DNA fragment that contains the binding site for the guide-RNA with a desired binding site for the guide-RNA.

14. The method of any of the preceding claims, comprising contacting the RNA-guided endonuclease with a target double-stranded DNA that is outside a cell.

15. The method of any of claims 1 to 13, comprising contacting the RNA-guided endonuclease with a target double-stranded DNA that is inside a test cell.

16. The method of any of preceding claims, wherein linking the first binding member of the specific binding pair to the chemoselective group is performed via a cycloaddition reaction.

17. The method of any of claims 1 to 10, further comprising quantifying the DNA fragment that contains the binding site for the guide-RNA.

18. The method of claim 17, wherein quantifying is performed using quantitative PCR.

19. The method of claim 15, further comprising:
(g) contacting an RNA-guided endonuclease without the guide RNA with the target double-stranded DNA inside a control cell, thereby producing ss-DNA at the site to which the guide-RNA binds,
(h) reacting the product of step (g) with kethoxal or an analog thereof modified by a chemoselective group, thereby adding the chemoselective group to any unpaired guanine base within the ssDNA,
(i) linking a first binding member of a second specific binding pair to the chemoselective group added in step (h) via a cycloaddition reaction,
(j) fragmenting the DNA after step (i) to produce fragmented target DNA from the control cell,
(k) enriching for fragments that contain the first binding member of the second specific binding pair by binding the product of step (j) to a support that comprises a second binding member of the second specific binding pair, which specifically binds to the first binding member of the second specific binding pair.

20. The method of claim 19, wherein the kethoxal or an analog thereof modified by a chemoselective group is N₃-kethoxal, and wherein the chemoselective group added to the unpaired guanine bases is azido.

21. The method of claim 19 or 20, wherein the first binding member of the second specific binding pair is a biotin moiety and the second binding member of the second specific binding pair is streptavidin.

22. The method of any of claims 19 to 21, further comprising:
(l) amplifying the enriched DNA from step (k).

23. The method of claim 22, wherein the method does not comprise releasing the DNA fragments comprising the first binding member of the second specific binding pair from the support after step (k), prior to step (l).

24. The method of claim 22 or 23, wherein step (l) comprises:
i. washing the support of (k) after the DNA fragments comprising the first binding member of the second specific binding pair have bound to the support; and then
ii. setting up an amplification reaction containing the support, without releasing from the support the DNA fragments comprising the first binding member of the second specific binding pair.

25. The method of any of claims 19 to 24, comprising sequencing the DNA fragment that contains the binding site for the guide-RNA.

26. The method of claim 25, wherein the sequencing is performed via a next generation sequencing method.

27. A kit comprising: 1) an RNA-guided endonuclease, 2) kethoxal or an analog thereof, modified by a chemoselective group, and 3) a first binding member of a specific binding pair having a functional group reactive to the chemoselective group of kethoxal or analog thereof.

28. The kit of claim 27, further comprising a support that comprises a second binding member of the second specific binding pair, which specifically binds to the first binding member of the second specific binding pair.

29. The kit of claim 26 or 27, wherein the first binding member of the specific binding pair is biotin moiety and the second binding member of the specific binding pair is streptavidin.
